(19) United States
(12) Patent Application Publication
(10) Pub. No.: US 2018/0250543 A1
(43) Pub. Date: Sep. 6, 2018

(54) PRESSURE BIOFEEDBACK DEVICE AND SYSTEM

(71) Applicant: MyCore Health, Inc.

(72) Inventor: Philip Alwitt, San Rafael, CA (US)

(21) Appl. No.: 15/910,353

(22) Filed: Mar. 2, 2018

Related U.S. Application Data
(60) Provisional application No. 62/466,947, filed on Mar. 3, 2017.

(51) Int. Cl. A63B 21/008; A63B 21/00

(52) U.S. Cl. CPC A63B 21/0085; A63B 21/00069; A61B 5/486; A63B 21/4025; A63B 21/4009

(57) ABSTRACT

The application discloses a pressure biofeedback system with improved safety, quality and precision.

### CROSS REFERENCE TO RELATED APPLICATIONS

This application claims priority from U.S. Provisional Application 62/466,947, filed on Mar. 3, 2017.

### BACKGROUND

This invention relates to exercise and monitoring devices particularly suited for use in physical therapy and physical fitness. In particular, the devices and systems disclosed herein are “pressure biofeedback” devices and systems designed to provide feedback that ensures safety, quality and precision in exercise performance and physical therapy.

Pressure biofeedback devices are known in the field of exercise and physiotherapy equipment. U.S. Pat. No. 5,338,276 to Jull et al. discloses an exercise monitoring device that includes an air bladder (or pressure pad), an air pump, an air line and feedback means to permit inflation of the air bladder for monitoring purposes. The bladder is placed between a body part of a patient (or subject) and a support surface, and monitors the pressure caused by the force of the body part .

Proper or ideal positioning of pressure biofeedback devices may be difficult while a patient or subject is preparing to perform the relevant exercise or physical therapy. Often, the subjects or patients are people with limited mobility such as the disabled or elderly. In such cases, positioning the device on the body before getting in position to exercise—most commonly in the supine position or leaning against a firm vertical surface such as a wall—is necessary.

### SUMMARY

There is a need for systems and devices that improve the safety, quality and precision of pressure biofeedback devices.

In one embodiment, there is provided a belt designed to hold a pressure biofeedback device in place, wherein the pressure biofeedback device comprises an air bladder or pressure pad, the belt comprising: (a) a pouch having a top, bottom and two sides, the pouch capable of receiving the air bladder or pressure pad of the pressure biofeedback device; (b) a first strap attached to a first side of the pouch; and (c) a second strap attached to a second side of the pouch, wherein the first and second straps comprise two parts of a fastening means that permits, when fastened, the belt to form a closed loop that is capable of fixing the pressure biofeedback device on a human body.

In another embodiment, there is provided a pressure biofeedback system comprising (a) a pressure biofeedback device comprising an air bladder or pressure pad; and (b) a belt designed to hold the device in place comprising a pouch having a top, bottom and two sides, the pouch capable of receiving the air bladder or pressure pad, a first strap attached to a first side of the pouch, and a second strap attached to a second side of the pouch, wherein the first and second straps comprise two parts of a fastening means that permits, when fastened, the belt to form a closed loop that is capable of fixing the device on a human body.

In still another embodiment, there is provided a pressure biofeedback system comprising a pressure feedback device comprising (a) an air bladder or pressure pad, the air bladder or pressure pad having two sides, (b) a first strap attached to a first side of the air bladder or pressure pad, and (c) a second strap attached to a second side of the air bladder or pressure pad, wherein the first and second straps comprise two parts of a fastening means that permits, when fastened, the belt to form a closed loop that is capable of fixing the device on a human body.

In yet another embodiment, there is provided in a method for strengthening muscles in a body part of a subject on a support surface, the method comprising the step of (a) placing a pressure biofeedback device that comprises an air bladder or pressure pad between the body part of the horizontal subject and the support surface, the improvement comprising the step of, prior to step (a), placing the device in the pouch of a belt, the belt comprising a pouch having a top, bottom and two sides, the pouch capable of receiving the air bladder or pressure pad of the pressure biofeedback device; a first strap attached to a first side of the pouch; and a second strap attached to a second side of the pouch, wherein the first and second straps comprise two parts of a fastening means that permits, when fastened, the belt to form a closed loop that is capable of fixing the pressure feedback device on a human body.

### BRIEF DESCRIPTION OF THE FIGURES

FIG. 1 is a perspective view of a pressure biofeedback system comprising a belt and a pressure biofeedback device capable of being removed from the belt pouch.

FIG. 2 is a perspective view of a pressure biofeedback system comprising a belt and a pressure biofeedback device fastened in place in the belt pouch.

FIG. 3 is a view of a horizontal subject with a pressure biofeedback system affixed to the subject's body.

FIG. 4 is a view of an upright subject leaning against a support surface with a pressure biofeedback system affixed to the subject's body.

FIG. 5 is a rear view of a vertical subject with a pressure biofeedback system affixed to the subject's body.

### DETAILED DESCRIPTION

As used herein, a “pressure biofeedback device” refers to a device having an air bladder or pressure pad. When the device has an air bladder, it also has a pressure gauge with an analog or digital readout, a connection between the air bladder and the pressure gauge which may consist of a tube or may be a direct connection between the two, a valve device to allow air to fill or release from the air bladder, and a pump to add air to the air bladder. The pressure gauge may be mechanical or digital. The pump may be hand operated or may be an automated pump such as an electric pump. When the device has a pressure pad, an electro-mechanical pressure pad may be substituted for the air bladder and gauge.

The belt described herein centers on a pouch into which the air bladder or pressure pad of the pressure biofeedback device can be inserted. The pouch is made of a flexible material such as any type of fabric or plastic film, or any material that will allow the air bladder or pressure pad to compress and alter the pressure as applied by the human body against a support surface. The belt will typically attach around the waist but may attach around the chest or neck as required to perform the intended exercise. The belt pouch may be open at the top or bottom or both. The pouch may include a flap to permit a full closure, or may employ other means of securing an air bladder or pressure pad to the belt such as snaps, Velcro™ or buttons. In one embodiment, the pouch may comprise a firm outer surface of the belt as the support surface.

The belt pouch is attached to two straps—one on each side of the pouch. The straps may consist of the same material as the pouch or may be a new material such as woven webbing. The strap material may be elastic to enhance user comfort while exercising. The straps may have a method of adjustment such as a cam buckle, tri-glide or Velcro™ to accommodate various body sizes. The straps may have a method to disengage such as a plastic or metal buckle or Velcro™ to enable simple donning and doffing of the belt. Alternatively, the two straps may be permanently linked, in which case they are essentially or in fact a single strap that forms a closed loop with the pouch. In this embodiment, the straps are preferably made of an elastic material to enable the closed loop belt to stretch when being put in place by the subject.

In another embodiment described herein, a belt pouch is not employed. Rather, the belt and straps described above are not attached to a belt pouch, but are attached directly to the air bladder or pressure pad.

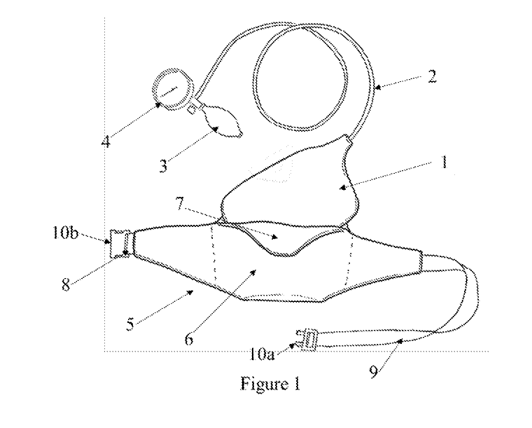

In FIG. 1, the pressure biofeedback device consisting of an air bladder [1] connected through air hose [2] to a pressure regulation system that includes air pump [3] and gauge [4] that acts as a feedback device. Also shown is the belt [5] comprising pouch [6], flap [7], a first strap [8] and second strap [9]. At the ends of each strap are portions [10a], [10b] that together buckle the straps to each other. The air bladder [1] is being inserted into open pouch [6].

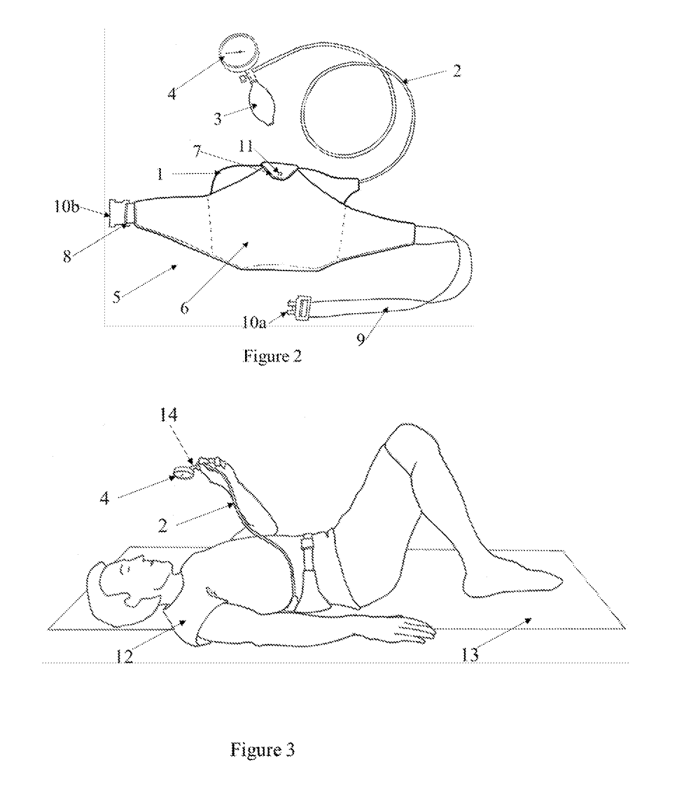

In FIG. 2, the pressure biofeedback device consisting of an air bladder [1] connected through air hose [2] to a pressure regulation system that includes air pump [3] and gauge [4] that acts as a feedback device. Also shown is the belt [5] comprising pouch [6], flap [7], a first strap [8] and second strap [9]. At the ends of each strap are portions [10a], [10b] that together buckle the straps to each other. The air bladder [1] is fully inserted into pouch [6], and the pouch is sealed with a snap fastener [11].

In FIG. 3, the air bladder has been inserted into the pouch of the belt which is then fastened around the midsection of a horizontal subject [12] to hold the air bladder in place between subject and support surface [13]. The air hose [2], pressure regulator [14] and gauge [4] are attached to the air bladder but remain outside the belt in order to be operated and viewed by the subject.

FIG. 3 also exemplifies the method for strengthening muscles in a body part of a subject on a support surface described herein. The belt fixes the air bladder in place between the body part and the support surface during exercise or therapy, thereby improving the accuracy, quality and safety of the system. In this figure, the subject and support surface are horizontal.

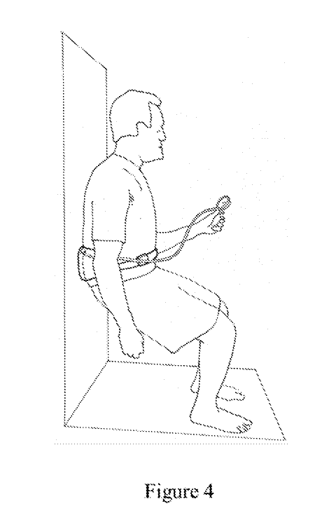

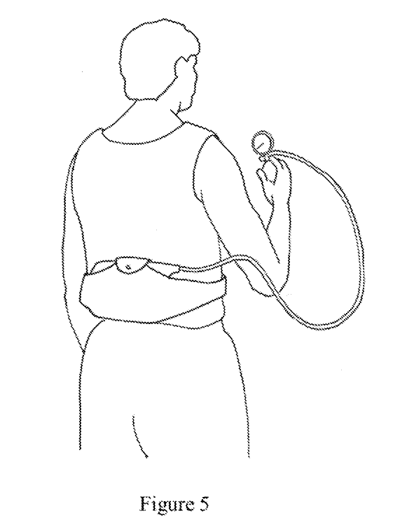

FIG. 4 shows an embodiment similar to FIG. 3, except the subject is near-vertical, and the bladder is disposed in the belt between the subject and a near-vertical support system. FIG. 5 depicts the rear view of a subject with the system in place.

While a number of exemplary embodiments, aspects and variations have been provided in the drawings and herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A belt designed to hold a pressure biofeedback device in place, wherein the pressure biofeedback device comprises an air bladder or pressure pad, the belt comprising:
(a) a pouch having a top, bottom and two sides, the pouch capable of receiving the air bladder or pressure pad of the pressure biofeedback device;
(b) a first strap attached to a first side of the pouch;
(c) a second strap attached to a second side of the pouch
wherein the first and second straps comprise two parts of a fastening means that permits, when fastened, the belt to form a closed loop that is capable of fixing the pressure feedback device on a human body.

2. The belt of claim 1 wherein the first or second strap further comprises a means of adjusting the length of said strap.

3. The belt of claim 2 wherein the adjusting means is elastic.

4. The belt of claim 1 wherein the pouch has an open top and a closed bottom that form a first pocket for receiving the air bladder or pressure pad.

5. The belt of claim 4 wherein the pouch further comprises a flap attached to the top of the pouch, the flap capable of fastening to the pouch to form a closed pocket.

6. The belt of claim 1 wherein the pressure biofeedback device further comprises a hose and a pressure regulator or gauge.

7. The belt of claim 6 wherein the pouch further comprises a second pocket for receiving the hose and the pressure regulator or gauge.

8. The belt of claim 6 wherein the first pocket is capable of receiving the entire pressure biofeedback device.

9. The belt of claim 1, wherein the means of fastening the first and second straps is accomplished by having a single strap function as both the first and second straps.

10. A pressure biofeedback system comprising:
(a) a pressure biofeedback device comprising an air bladder or pressure pad; and
(b) a belt designed to hold the device in place comprising a pouch having a top, bottom and two sides, the pouch capable of receiving the air bladder or pressure pad, a first strap attached to a first side of the pouch, and a second strap attached to a second side of the pouch, wherein the first and second straps comprise two parts of a fastening means that permits, when fastened, the belt to form a closed loop that is capable of fixing the device on a human body.

11. The system of claim 10 wherein the pressure biofeedback device further comprises a hose and a pressure regulator or gauge.

12. The system of claim 10 wherein the air bladder or pressure pad is permanently affixed to the pouch.

13. The system of claim 10 wherein the air bladder or pressure pad is removable from the pouch.

14. A pressure biofeedback system comprising a pressure feedback device comprising:
(a) an air bladder or pressure pad, the air bladder or pressure pad having two sides;
(b) a first strap attached to a first side of the air bladder or pressure pad; and
(c) a second strap attached to a second side of the air bladder or pressure pad, wherein the first and second straps comprise two parts of a fastening means that permits, when fastened, the belt to form a closed loop that is capable of fixing the device on a human body.

15. In a method for strengthening muscles in a body part of a subject on a support surface, the method comprising the step of (a) placing a pressure biofeedback device that comprises an air bladder or pressure pad between the body part of the subject and the support surface, the improvement comprising the step of, prior to step (a), placing the device in the pouch of a belt of claim 1.

16. The method of claim 15 wherein the subject is a horizontal subject and the support surface is a horizontal support surface.

17. The method of claim 15 wherein the subject is a non-horizontal subject and the support surface is a non-horizontal support surface.
